(19) United States
(12) Patent Application Publication
(10) Pub. No.: US 2025/0000678 A1
(43) Pub. Date: Jan. 2, 2025

(54) APPARATUSES AND METHODS FOR EXPANDABLE BRANCHING STENT PROSTHESES

(71) Applicant: Merit Medical Systems, Inc., South Jordan, UT (US)

(72) Inventors: Michael Adams, Bluffdale, UT (US); Joanne Hislop, American Fork, UT (US); Jason Wiersdorf, West Jordan, UT (US); Yuma Medina, South Jordan, UT (US)

(21) Appl. No.: 18/753,791

(22) Filed: Jun. 25, 2024

Related U.S. Application Data
(60) Provisional application No. 63/511,139, filed on Jun. 29, 2023.

(51) Int. Cl. A61F 2/954; A61F 2/958; A61M 25/00; A61M 25/09; A61M 25/10

(52) U.S. Cl. CPC A61F 2/954; A61F 2/958; A61M 25/0026; A61M 25/09; A61M 2025/1015; A61M 2025/1061

(57) ABSTRACT

Expandable branching stent prostheses and methods of deployment for the same are discussed herein. A deployment system includes an expandable branching stent prosthesis having a trunk portion, a first branching portion, and a second branching portion. The deployment system also includes a first balloon disposed within the first branching portion and the trunk portion, the first balloon being configured to inflate to deploy the first branching portion and the trunk portion of the expandable branching stent prosthesis. The deployment system also includes a second balloon disposed within the second branching portion and configured to inflate to deploy the second branching portion of the expandable branching stent prosthesis.

### RELATED APPLICATIONS

This application claims priority to U.S. Provisional Application No. 63/511,139, filed on Jun. 29, 2023 and titled, “Apparatuses and Methods for Expandable Branching Stent Prosthesis,” which is hereby incorporated by reference in its entirety.

### TECHNICAL FIELD

The present application relates to medical devices including stent prostheses, deployment devices, and methods of using the same.

### BRIEF DESCRIPTION OF THE SEVERAL VIEWS OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 illustrates a perspective view of an expandable branching stent prosthesis, according to embodiments herein.

FIG. 2 illustrates a deployed expandable branching stent prosthesis, according to embodiments herein.

FIG. 3 illustrates a deployment system configured for use with an expandable branching stent prosthesis, according to embodiments herein.

FIG. 4 illustrates a cross-sectional view of a catheter of the deployment system of FIG. 3.

FIG. 5 illustrates a first deployment stage for a deployment system used to deploy an expandable branching stent prosthesis at a branching implant site, according to embodiments herein.

FIG. 6A illustrates a subsequent deployment stage for the deployment system, according to embodiments herein.

FIG. 6B illustrates another subsequent deployment stage for the deployment system, according to embodiments herein.

FIG. 7 illustrates a method according to embodiments disclosed herein.

### DETAILED DESCRIPTION

Delivery catheter systems may be configured to deliver one or more medical appliances or systems to a location within a patient's body and deploy the medical appliance or system within the patient's body. For example, such a delivery catheter system may be configured to be advanced from an insertion site at the outside of an anatomical system to a treatment location within the anatomical system. For example, a delivery catheter system may be configured to be advanced through bends, turns, or other structures within the anatomy of the vasculature.

A stent prosthesis may be disposed within a portion of the delivery catheter system (e.g., as or as part of a medical appliance or system) such that a practitioner may deploy the stent prosthesis from a distal end of the delivery catheter system through manipulation of one or more components of a handle assembly of the delivery catheter system.

Stent prostheses may be deployed in various body lumens for a variety of purposes. Stent prostheses may be deployed, for example, in the arterial system for a variety of therapeutic purposes including the treatment of occlusions within the lumens of that anatomical system. It will be appreciated that the current disclosure may be applicable to stent prostheses designed for the central venous system, peripheral vascular system, abdominal aortic aneurism treatment, bronchial system, esophageal system, biliary system, or any other system of the human body. Further, the present disclosure may equally be applicable to other prosthesis such as grafts.

Accordingly, it will be understood that while specific examples recited herein may refer to deployment of cardiovascular stent prostheses within a cardiovascular system, analogous concepts and devices may be used in/with various other anatomical systems of the body, including for placement and deployment of medical appliances in the gastrointestinal tract (including, for example, within the esophagus, intestines, stomach, small bowel, colon, and biliary duct); the respiratory system (including, for example, within the trachea, bronchial tubes, lungs, nasal passages, and sinuses); or any other location within the body, both within bodily lumens (for example, the ureter, the urethra, etc.) and within other bodily structures.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “proximal” and “distal” are used herein to refer to opposite locations relative to a medical device in use by a practitioner. The proximal end of the device is defined as the end of the device closest to the practitioner when the device is in use by the practitioner. The distal end is the end opposite the proximal end.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

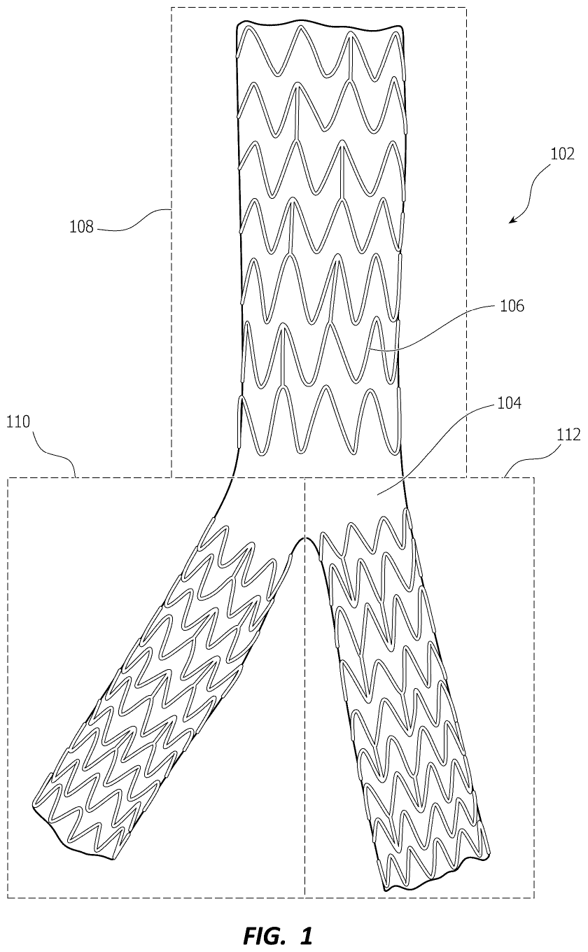

FIG. 1 illustrates a perspective view of an expandable branching stent prosthesis 102, according to embodiments herein. The expandable branching stent prosthesis 102 of FIG. 1 is illustrated in a deployed (expanded) position.

In the illustrated embodiment, the expandable branching stent prosthesis 102 is partially composed of a wire or laser cut stent 106. The wire or laser cut stent 106 is configured to be expandable to the illustrated deployed position from an unexpanded/un-deployed configuration (e.g., via the inflation of one or more balloons from within the expandable branching stent prosthesis 102) after the expandable branching stent prosthesis 102 is delivered to a desired location. The wire or laser cut stent 106 may be formed of any suitable material, such as nickel-titanium alloy, stainless steel, cobalt-chromium, platinum, polymers, etc. The wire or laser cut stent 106 may have a zig-zag pattern, a wave pattern, or any other suitable pattern. The wire or laser cut stent 106 may be pre-formed or formed corresponding to a tubular body 104. The material, pattern, and wire diameter of a wire or laser cut stent 106 that is a wire stent, or the wall thickness and strut width of a wire or laser cut stent 106 that is a laser cut stent, may be configured to provide a chronic radial outwardly directed force and a resistance to a radial inwardly directed force. For a non-self-expanding design the deployed wire or laser cut stent 106 may be configured to provide a radial stiffness and radial strength to resist a local or radial inwardly directed force.

The expandable branching stent prosthesis 102 further includes the tubular body 104. The tubular body 104 may be formed of a variety of materials and/or layers of materials, including biocompatible materials that are resistant to passage of fluid through a wall of the tubular body 104. For example, the tubular body 104 may be formed of polyethylene terephthalate, polyurethane, silicone rubber, nylon, fluoropolymer, polyester, etc. A thickness of the wall may range from about 0.025 mm to about 0.5 mm.

In certain embodiments, the wall of the tubular body 104 may be impermeable to tissue cell ingrowth into and/or tissue cell migration through the wall, for example, to prevent or discourage stenosis of the tubular body 104. Additionally or alternatively, in some embodiments, the wall of the tubular body 104 can be impermeable to fluid such that fluid is prevented from leaking from the inside of the expandable branching stent prosthesis 102 to the exterior of the expandable branching stent prosthesis 102 and into surrounding tissue. In some embodiments an interior surface of the wall may include serially deposited fibers of polytetrafluoroethylene (PTFE) to resist fibrin deposition and platelet adhesion on the surfaces.

Note that in embodiments herein, stent prostheses are illustrated as having tubular bodies (such as the tubular body 104 of FIG. 1). However, it will be understood that expandable branching stent prostheses using a wire scaffold, framework, or stent without cover or other tubular body fall within the scope of the disclosure.

As illustrated herein, the expandable branching stent prosthesis 102 may be for deployment at a branching implant site within a body having a trunk, a first branch, and a second branch. Accordingly, the expandable branching stent prosthesis 102 includes a trunk portion 108, a first branching portion 110, and a second branching portion 112. For deployment to the expandable branching stent prosthesis 102 at the branching implant site, the trunk portion 108 of the expandable branching stent prosthesis 102 is used in/at the trunk of the branching implant site, the first branching portion 110 is deployed in/at the first branch of the branching implant site, and the second branching portion 112 is deployed in/at the second branch of the branching implant site.

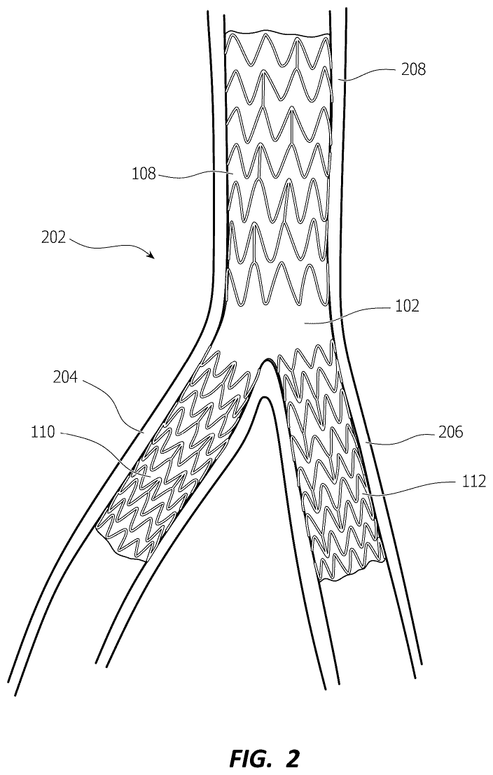

FIG. 2 illustrates a deployed expandable branching stent prosthesis 102, according to embodiments herein. In this expanded/deployed configuration, the expandable branching stent prosthesis 102 provides stenting at/through a branching implant site 202 within the anatomical system in which the expandable branching stent prosthesis 102 is deployed.

The branching implant site 202 may be a portion of an anatomical system that includes the first branch 204, the second branch 206, and the trunk 208 (and that, e.g., communicates fluids through these). In some cases, the branching implant site 202 may be the aorto-illiac bifurcation in the arterial system. In the illustrated deployed configuration, the trunk portion 108 of the expandable branching stent prosthesis 102 is deployed at/within the trunk 208 of the branching implant site 202, the first branching portion 110 of the expandable branching stent prosthesis 102 is deployed at/within the first branch 204 of the branching implant site 202, and the second branching portion 112 of the expandable branching stent prosthesis 102 is deployed at/within the second branch 206 of the branching implant site 202.

In some embodiments, the expandable branching stent prosthesis 102, once deployed, provides an appropriate channel for desired liquid flow through the branching implant site 202. It may be that the region around the branching implant site 202 is diseased, misshapen, and/or damaged, and that the deployment of the expandable branching stent prosthesis 102 in the illustrated manner can correct and/or ameliorate attendant issues.

As illustrated, the branching implant site 202 of FIG. 2 is used within an anatomical system that is a cardiovascular system. However, it should be noted that while examples herein describe (and figures herein illustrate) the deployment of expandable branching stent prostheses within cardiovascular systems, the cardiovascular anatomical context is given by way of example and not by way of limitation. It will be understood that deployment systems analogous to those described in relation to disclosure herein may be used to deploy appropriate expandable branching stent prostheses with/at branching implant sites of other anatomical systems, and that corresponding methods for using such deployment systems to deploy corresponding expandable branching stents that are analogous to those methods described herein could be used in those other anatomical contexts.

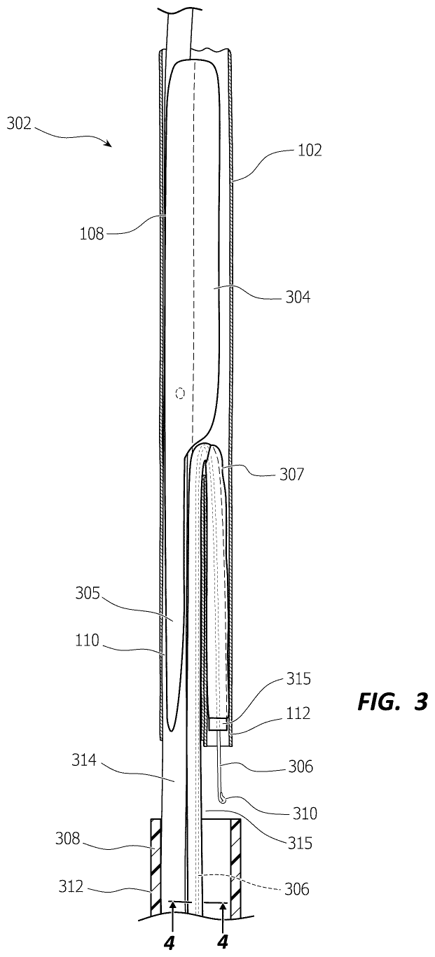

FIG. 3 illustrates a deployment system 302 configured for use with an expandable branching stent prosthesis 102, according to embodiments herein. As illustrated, the deployment system 302 may include an expandable branching stent prosthesis 102, a first balloon 304, a second balloon 307, and a floss wire 306. In many embodiments, the deployment system 302 also may include a first catheter 314 secured or securable to the first balloon 304 and a second catheter 315 secured or securable to the second balloon 307. Each of the first catheter 314 and the second catheter 315 may include one or more inflation lumens in fluid communication with the respective first balloon 304 or second balloon 307 to selectively inflate and/or deflate the respective first balloon 304 or second balloon 307.

The expandable branching stent prosthesis 102 is illustrated in FIG. 3 in a collapsed (non-deployed configuration), but is otherwise as described in relation to FIG. 1. A delivery catheter system 308 may be used to deliver the deployment system 302 to a desired location. The deployment system 302 may be mounted on the first catheter 314 of the delivery catheter system 308 by passing the first catheter 314 through the first branching portion 110 and the trunk portion 108 of the expandable branching stent prosthesis 102. A catheter sleeve 312 of the deployment system 302 may initially cover/surround the deployment system 302 in order to hold the deployment system 302 in place on the first catheter 314 and the second catheter 315, and to prevent snagging of the deployment system 302 during placement. Once the deployment system 302 has been located in the desired location, the catheter sleeve 312 may be retracted back along the first catheter 314 and the second catheter 315 to expose the deployment system 302 (as illustrated in FIG. 3).

In alternative cases, it may be that the deployment system 302 is not covered by the catheter sleeve 312 during placement. In such a case, the first catheter 314 on which the deployment system 302 is mounted (e.g., via crimping) would be manipulated to extend outward from the delivery catheter system 308 until the first catheter 314 is located at the desired location (as illustrated in FIG. 3).

The first balloon 304 is arranged within the expandable branching stent prosthesis 102. The first balloon 304 may be attached to the first catheter 314 along a length of the first balloon 304, and may be inflated and/or deflated at the option of a practitioner operating the delivery catheter system 308 via a connection between the first balloon 304 and an inflation lumen of the catheter 314 that communicates fluid between the inflation lumen of the first catheter 314 and the first balloon 304. The first catheter 314 also may include a guide wire or stiffener wire extending therethrough (e.g., extending through a wire lumen in the first catheter 314.

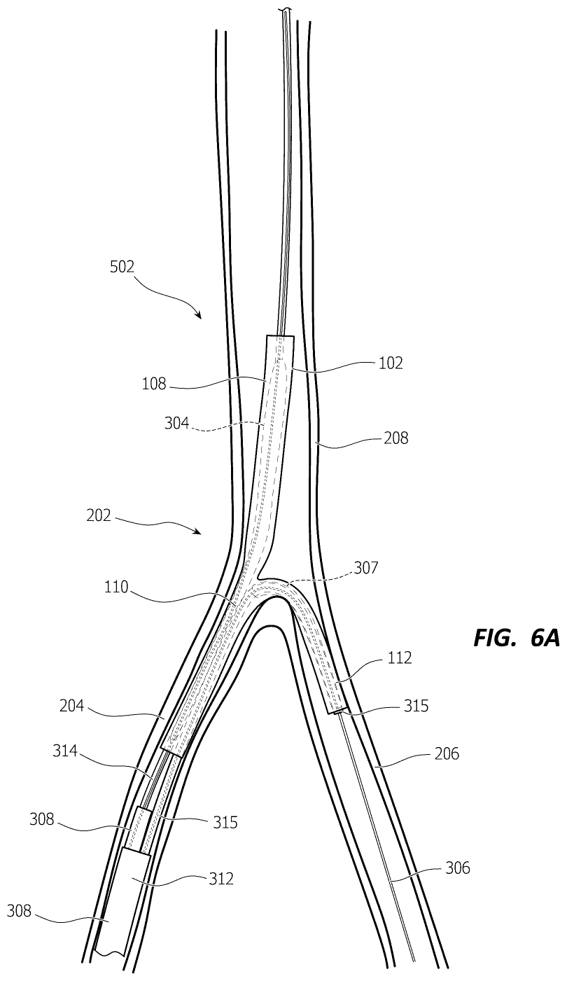

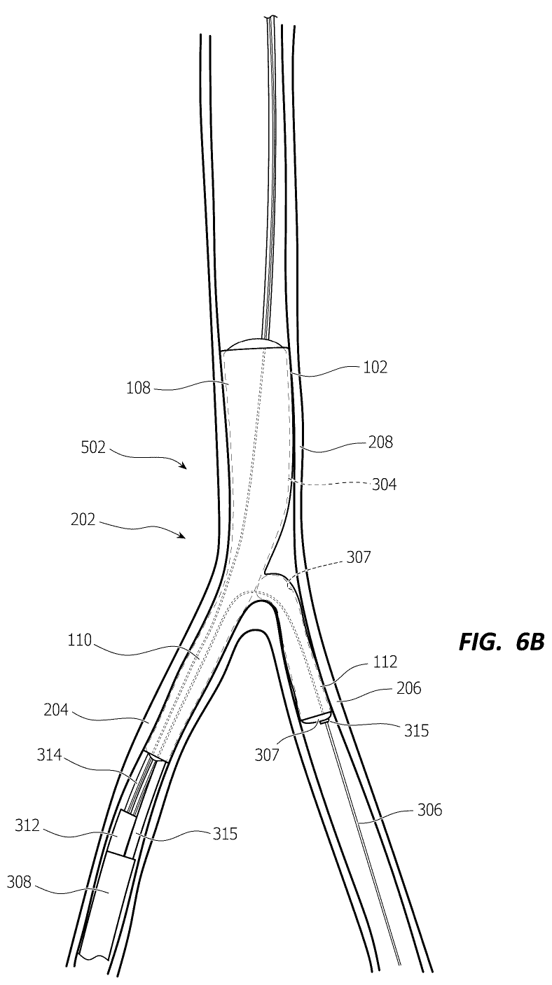

As shown in FIG. 3, due to the shape of the first balloon 304 and its placement/arrangement within the expandable branching stent prosthesis 102, when the first balloon 304 is inflated, the first balloon 304 expands the trunk portion 108 and the first branching portion 110 of the expandable branching stent prosthesis 102 for deployment. In some embodiments, the first balloon 304 includes a first balloon portion 305 disposed within the first branching portion 110 and a trunk balloon portion 303 disposed within the trunk portion 108 of the expandable branching stent prosthesis 102 for deployment. The trunk balloon portion 303 of the first balloon 304 may be sized differently (e.g. larger) than the first balloon portion 305 of the first balloon 304. For example, when the first balloon 304 is inflated (see FIG. 6B), the first balloon portion 305 disposed in the first branching portion 110 may include a first diameter or a first lateral width, and the trunk balloon portion 303 disposed in the trunk portion 108 may include a trunk diameter or a trunk lateral width greater than the first diameter or the first lateral width of the first balloon portion 305 disposed in the first branching portion 110.

The second balloon 307 is separate and distinct from the first balloon 304, and arranged or otherwise disposed within the second branching portion 112 of the expandable branching stent prosthesis 102. The second balloon 307 may be inflated and/or deflated at the option of a practitioner operating the delivery catheter system 308 via a connection between the second balloon 307 and an inflation lumen of the second catheter 315 that communicates fluid between the inflation lumen of the second catheter 315 and the second balloon 307 (e.g., inflation of the second balloon 307 may be exclusively through the second catheter 315). In some embodiments, while the second balloon 307 is separate and distinct from the first balloon 304, the inflation lumens of the first catheter 314 and the second catheter 315 may be fluidly connected or coupled (e.g., with a fitting) such that the first balloon 304 and the second balloon 307 inflate substantially simultaneously (e.g., at the same time). In these and other embodiments, both of the inflation lumens of the first catheter 314 and the second catheter 315 may extend from or be secured to the same hub (e.g., proximal hub) of the deployment system. In some embodiments, the inflation lumens of the first catheter 314 and the second catheter 315 are separate.

As can be seen, due to the shape of the second balloon 307 and its placement/arrangement within the expandable branching stent prosthesis 102, when the second balloon 307 is inflated, the second balloon 307 expands the second branching portion 112 of the expandable branching stent prosthesis 102 for deployment. The trunk balloon portion 303 of the first balloon 304 may be sized differently (e.g., larger) than the second balloon 307. For example, when the first balloon 304 and the second balloon 307 are inflated (see FIG. 6B), the second balloon 307 may include a second diameter or a second width that is less than the trunk diameter or the trunk lateral width of the trunk balloon portion 303. In some embodiments, the second balloon 307 and the first balloon portion 305 may include a similar or substantially equal diameter or lateral width upon inflation (e.g., the first diameter or the first lateral width of the first balloon portion 305 may be similar or substantially equal to the second diameter or the second lateral width of the second balloon 307 upon inflation of the first balloon 304 and the second balloon 307).

The second balloon 307 may be attached to the second catheter 315 along a length of the second catheter 315. In some embodiments, the second catheter 315 terminates at the second balloon 307, and does not extend along the length of the second balloon 307. The second catheter 315 may be disposed in the expandable branching stent prosthesis 102 external or outside of the first balloon 304. In some embodiments, the second catheter 315 extends through the first branching portion 110 of the expandable branching stent prosthesis 102 between the first balloon portion 305 of the first balloon 304 and the inner surface of the first branching portion 110 of the expandable branching stent prosthesis 102. The second catheter 315 may then bend to be oriented to or disposed at least partially in the second branching portion 112 of the expandable branching stent prosthesis 102. In some embodiments, the second catheter 315 bends to extend over the intersection of the first branching portion 110 and the second branching portion 112, and the second catheter 315 may extend at least partially into the second branching portion 112. This configuration of the second catheter 315 extending through first branching portion 110 and at least partially into the second branching portion 112 provides for less movement of the second balloon 307 in the second branching portion 112 when the balloons 304, 307 and/or the catheters 314, 315 are removed. In some embodiments, the second catheter 315 terminates at least proximate to the second balloon 307 and the second catheter 315 does not bend to extend over the intersection of the first branching portion 110 and the second branching portion 112. Instead, an end region (e.g., the cone of the second balloon 307) may bend to extend over the intersection of the first branching portion 110 and the second branching portion 112, and the second catheter 315 may terminate in the first branching portion 110 (e.g., the second catheter does not extend into the second branching portion 112).

In some embodiments, the first catheter 314 and second catheter 315 are connected such that when one catheter (of the first catheter 314 and the second catheter 315) is removed from implant site 202 the other catheter (of the first catheter 314 and the second catheter 315) is simultaneously removed. In some embodiments, the second catheter 315 may not be connected to the first catheter 314 at any point along first catheter 314, or may be selectively connected and disconnectable. When the second catheter 315 is disconnected from the first catheter 314, the first catheter 314 and the second catheter 315 may be removed non-simultaneously (e.g., at different times) from the implant site 202. A lower removal force may be used to remove the first catheter 314 and the second catheter 315 when disconnect than when connected.

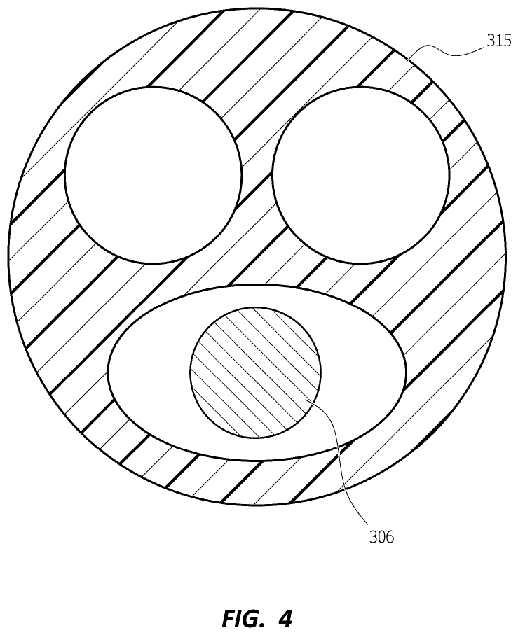

In some embodiments, at least a portion of the floss wire 306 extends through the second catheter 315. FIG. 4 is a cross-sectional view of the second catheter 315, according to an embodiment. The second catheter 315 may include a wire lumen 406 sized and dimensioned to allow the floss wire 306 to extend therethrough. As noted above, the second catheter 315 can also include an inflation lumen configured to selectively inflate the second balloon 307. In some embodiments, the second catheter 315 may include multiple inflation lumens 410 in fluid communication with the second balloon 307 and configured to selectively inflate the second balloon 307.

While a floss wire 306 is referenced herein, in some embodiments, the floss wire 306 includes and/or is used in connection with an additional stiffener wire or through wire. The floss wire 306, and/or a stiffener wire, may be configured to prevent or inhibit kinking or closing of the multiple inflation lumens 410 as the catheter 315 bends around the bifurcated intersection of the first branching portion 110 and the second branching portion 112 of the expandable branching stent prosthesis 102. Nonetheless, multiple inflation lumens 410 are provided, according to an embodiment, such that in the even a first inflation lumen of the multiple inflation lumens 410 kinks to disrupt the flow of fluid therethrough, a second inflation lumen of the multiple inflation lumens 410 is present and in fluid communication with the second balloon 307 such that the second balloon 307 may still be inflated even with the disrupted flow of fluid in the first inflation lumen. In other words, the multiple inflation lumens 410 may be configured wherein one or more lumens are redundant to aid in inflation when the device is disposed within tortuous pathways.

In some embodiments, the distal end region of the floss wire 306, disposed in the wire lumen 406, passes through the first branching portion 110 of the expandable branching stent prosthesis 102. The distal end region of the floss wire 306 may then extend through and out the second branching portion 112 of the expandable branching stent prosthesis 102, as illustrated. In some embodiments, the distal end region of the floss wire 306 extends through and out of the second balloon 307 such that the second balloon 307 is concentric with the distal end region of the floss wire 306. In some embodiments, the second catheter 315, having the distal end region of the floss wire 306 disposed therein, may extend through and out of the second balloon 307 such that the second balloon 307 is concentric with the second catheter 315 and the distal end region of the floss wire 306. The distal end region of the floss wire 306 may terminate in a catch feature 310 that is configured to interact with a snare feature of a snare wire, in the manner described herein.

When deployed within an anatomical system, the floss wire 306 may extend back through a branch of a branching implant site for planned deployment of the expandable branching stent prosthesis 102 of the deployment system 302 and run to/through an insertion site through which a practitioner has delivered the deployment system 302 to the body (e.g., using a delivery system (such as, e.g., a delivery catheter system) that includes the deployment system 302).

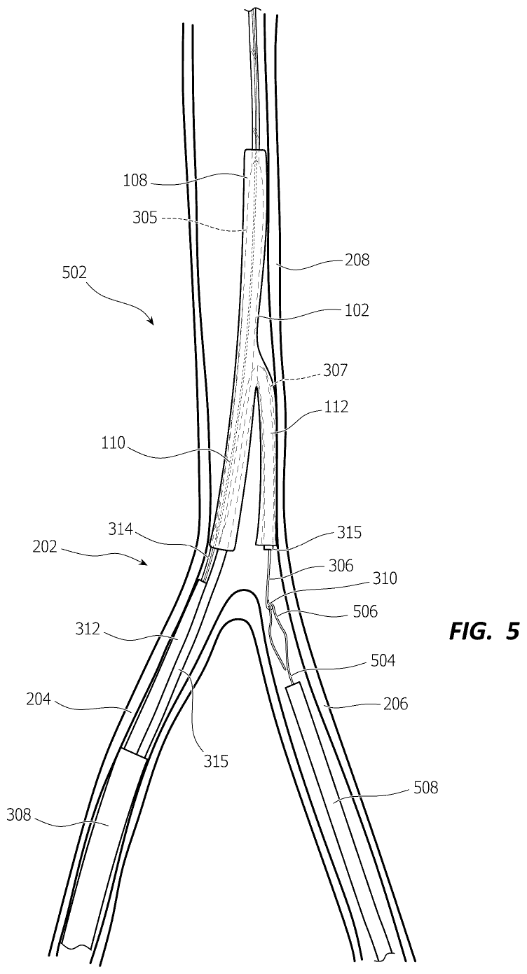

Methods for using deployment systems described herein to deploy expandable branching stent prostheses to a branching implant site within a body are now described. FIG. 5 illustrates a first deployment stage for a deployment system 302 used to deploy an expandable branching stent prosthesis 102 at a branching implant site 202, according to embodiments herein. One or more incisions (not illustrated) giving access to a first branch 204 of the branching implant site 202 is made in the body. Then, the delivery catheter system 308 is used to deliver, via the first branch 204, the deployment system 302 to the illustrated location past the first branch 204 and into the trunk 208 of the branching implant site 202.

While advancing up through the first branch 204, the deployment system 302 may be covered by the catheter sleeve 312 of the delivery catheter system 308 to facilitate positioning within the trunk 208. Then, once the desired positioning of the deployment system 302 is achieved, the catheter sleeve 312 may be retracted along the first catheter lumen 314 and the second catheter lumen 315 to expose the deployment system 302 at a selected position (e.g., such that the positioning of the deployment system 302 is as illustrated in FIG. 5, but prior to the use of the snare wire 504 to snare the floss wire 306, to be described below).

Alternatively, the catheter lumen 314 may be advanced forward from the delivery catheter system 308 until the deployment system 302 is located in the desired position (e.g., such that the positioning of the deployment system 302 is as illustrated in FIG. 5 (but prior to the use of the snare wire 504 to snare the floss wire 306, to be described below).

As illustrated, the deployment system 302 is delivered with an orientation on the catheter lumen 314 such that the first branching portion 110 and the second branching portion 112 of the expandable branching stent prosthesis 102 are oriented corresponding to the first branch 204 and the second branch 206 of the branching implant site 202.

It is also noted that the floss wire 306 may extend back from the deployment system 302 and to the insertion site corresponding to the incision(s) for access to the first branch 204. Once the deployment system 302 is in the desired position, one or more incisions (not illustrated) giving access to the second branch 206 of the branching implant site 202 is made in the body. Then, a distal end of a snare wire 504 may be delivered to the branching implant site 202 via the second branch 206 (e.g., using a second delivery catheter system 508 through the access incision(s) for the second branch 206). The snare wire 504 may extend back through the second branch 206 of the branching implant site 202 for to/through an insertion site corresponding to the incision(s) for access to the second branch 206.

As illustrated, the distal end of the snare wire 504 includes a snare feature 506 that is configured to interact with the catch feature 310 of the floss wire 306 in order to connect and/or snare the distal end of the floss wire 306 together with the distal end of the snare wire 504 at the branching implant site 202. A practitioner manipulating the floss wire 306 and/or the snare wire 504 from their respective corresponding insertion sites may cause this connecting and/or snaring together to occur.

FIG. 6A and FIG. 6B illustrate further subsequent deployment stages for the deployment system 302, according to embodiments herein. According to the embodiments of FIG. 6A and FIG. 6B, after the floss wire 306 and the snare wire 504 are connected together (as illustrated in FIG. 5), the snare wire 504 may be pulled back along the second branch 206 in order to bring the distal end of the floss wire 306 through the second branch 206 and out through the insertion site/incision for the access to the second branch 206, as has been described. FIG. 6A accordingly illustrates that the floss wire 306 has been pulled through in this manner.

FIG. 6A also illustrates an example positioning of the deployment system 302 once a desired seating of the expandable branching stent prosthesis 102 has occurred. The pulling of the floss wire 306 through the second branch 206 may cause the first branching portion 110 of the expandable branching stent prosthesis 102 to become seated along the first branch 204 of the branching implant site 202 and the second branching portion 112 of the expandable branching stent prosthesis 102 to become seated along the second branch 206 of the branching implant site 202. Note that if necessary, after the distal end of the floss wire 306 is exposed to the practitioner through the insertion site/incision for the access to the second branch 206, further manipulation of the expandable branching stent prosthesis 102 may be performed using both ends of the floss wire 306 at the respective insertion site for each end in order to achieve the appropriate seating of the first branching portion 110 and the second branching portion 112 of the expandable branching stent prosthesis 102 in this manner. For example, the snare wire 504 and/or the floss wire 306 may be pulled to move the second branching portion 112 further into the second branch 206 of the branching implant site 202 than is shown in FIG. 6A. In some embodiments, the snare wire 504 and/or the floss wire 306 may be pulled to move the second branching portion 112 further into the second branch 206 of the branching implant site 202 until the second catheter is 315 is position over the bifurcated intersection of the first branch 204 and the second branch 206 of the branching implant site 202 and the second balloon 307 is disposed substantially entirely in the second branch 206 of the branching implant site 202. In this positioning, the second catheter 315 may bend to extend over the intersection of the first branching portion 110 and the second branching portion 112 of the branching stent prosthesis, and the second catheter 315 may extend at least partially into the second branching portion 112.

With the deployment system 302 in the illustrated position, it may be that the expandable branching stent prosthesis 102 of the deployment system 302 can be usefully deployed within the branching implant site 202. Turning to FIG. 6B, with the expandable branching stent prosthesis 102 appropriately seated (e.g., as was discussed in FIG. 6A), the first balloon 304 and the second balloon 307 of the deployment system 302 may be inflated (e.g., the practitioner may manipulate the delivery catheter system 308 for the balloon 304 to cause the inflation of the first balloon 304 and the second balloon 307). Inflation of the first balloon 304 and the second balloon 307 may be substantially simultaneous. The inflation of the first balloon 304 expands the trunk portion 108 and the first branching portion 110 of the expandable branching stent prosthesis 102, such that the trunk portion 108 and the first branching portion 110 deploy against the walls of the trunk 208 and the first branch 204, respectively, of the branching implant site 202, as illustrated in FIG. 6B. This inflation of the second balloon 307 expands the second branching portion 112 of the expandable branching stent prosthesis 102, such that the second branching portion 112 deploys against the walls of the second branch 206 of the branching implant site 202, as also illustrated in FIG. 6B.

With the expandable branching stent prosthesis 102 deployed, the first balloon 304 and the second balloon 307 may be at least partially (e.g., entirely) deflated. In some embodiments, the first balloon 304 and the second balloon 307 may be deflated via the inflation lumens of the first catheter 314 and the second catheter 315, respectively. In some embodiments, a vacuum source may be used to deflate the first balloon 304 and the second balloon 307.

In some embodiments, the floss wire 306 may then be removed from the deployment system 302. For example, with the catch feature 310 and the snare feature 506 snared together, the floss wire 306 may be pulled by the snare wire 504 to pull the floss wire through the second catheter 315, through the second balloon 307, and out the insertion site for the snare wire 504. In some embodiments, the floss wire 306 may be removed by pulling the floss wire 306 through the second balloon 307, and through at least some (e.g., all) of the second catheter 315. As noted above, in some embodiments (not shown), the second catheter 315 terminates proximate to the second balloon 307 (e.g., the second catheter does not extend through the second balloon 307, and the second balloon 307 does not include a through lumen). In these and other embodiments, the floss wire 306 may extend through the wire lumen 406 of the second catheter 315 (shown in FIG. 4) and exit the second catheter 315 before the second catheter 315 attaches to the second balloon 307. The floss wire 306 may then extend through the second branching portion 112 between the second balloon 307 and the second branching portion 112.

The delivery system 302 may then be removed by pulling on at least one (e.g., both) of the first catheter 314 and the second catheter 315 (or a device secured to the first catheter 314 and the second catheter 315). This pulling directs this second balloon 307, now deflated, over the bifurcated intersection of the first branching portion 110 and the second branching portion 112 of the expandable branching stent prosthesis 102. In conventional systems, setting of the second branching portion 112 with a balloon and removing the balloon used to deploy the second branching portion 112 often pulled the balloon too far out of the second branching portion 112, thereby disrupting placement of the expandable branching stent prosthesis 102. In the delivery system 302, however, the second balloon 307 extends very minimally, if at all, out of the second branching portion 112, and is more easily removed by pulling the second balloon 307 over the bifurcation and then through the first branching portion 110. The delivery system 302 may then be removed away from the branching implant site 202 and the body altogether through an introducer sheath. In some embodiments, the catheter sleeve 312 may be used to assist in removing the delivery system 302 away from the branching implant site 202 and the body altogether. For example, the first catheter 314 and the second catheter 315 may be secured to the catheter sleeve 312 such that pulling the catheter sleeve 312 pulls the first catheter 314 (and the first balloon 304) and the second catheter 315 (and the second balloon 307).

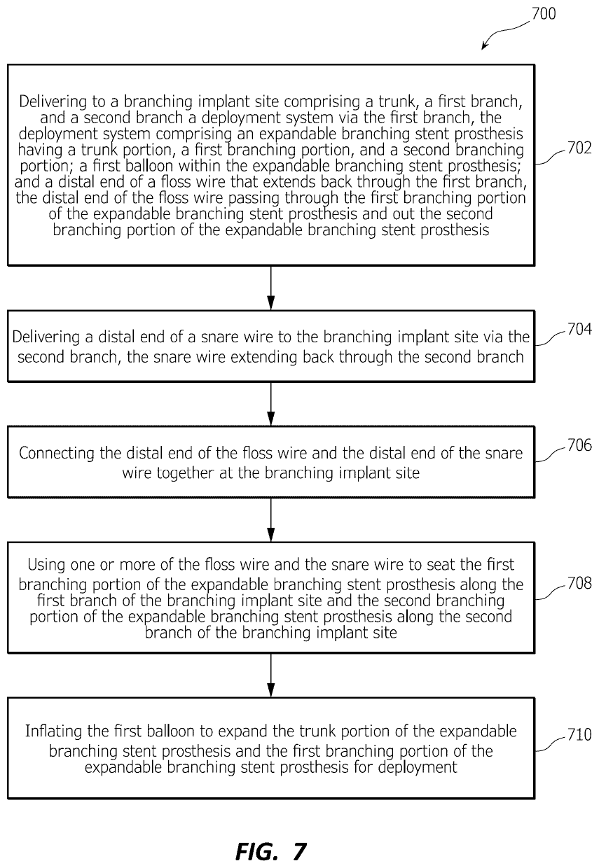

FIG. 7 illustrates a method 700, according to embodiments disclosed herein. The method 700 includes delivering 702 to a branching implant site comprising a trunk, a first branch, and a second branch, a deployment system via the first branch. The deployment system may include any deployment system described herein. In some embodiments, the deployment system includes: an expandable branching stent prosthesis having a trunk portion, a first branching portion, and a second branching portion; a first balloon disposed within the first branching portion and the trunk portion of the expandable branching stent prosthesis; a second balloon disposed within the second branching portion; and a floss wire passing through the first branching portion and out the second branching portion of the expandable branching stent prosthesis. The floss wire may comprise a distal end disposed outside of the second branching portion of the expandable branching stent prosthesis. In some embodiments, delivering 702 the deployment system includes delivering 702, via the first branch, the deployment system in a catheter sleeve surrounding the expandable branching stent prosthesis, the first balloon, the second balloon, the first catheter, and the second catheter during the delivery. The branching implant site may be within a vascular system.

The method 700 further includes delivering 704 a distal end of a snare wire to the branching implant site via the second branch the snare wire extending back through the second branch.

The method 700 further includes connecting 706 the distal end of the floss wire and the distal end of the snare wire together at the branching implant site. In some embodiments, connecting 706 the distal end of the floss wire and the distal end of the snare wire together at the branching implant site includes connecting 706 the distal end of the snare wire to a catch at the distal end of the floss wire.

The method 700 further includes using 708 one or more of the floss wire and the snare wire to seat the first branching portion of the expandable branching stent prosthesis along the first branch of the branching implant site and the second branching portion of the expandable branching stent prosthesis along the second branch of the branching implant site.

The method 700 further includes inflating 710 the first balloon to expand the trunk portion of the expandable branching stent prosthesis and the first branching portion of the expandable branching stent prosthesis for deployment. In some embodiments inflating 710 the first balloon includes inflating 710 the first balloon with a first catheter coupled to the first balloon and having a first inflation lumen in fluid communication with the first balloon to expand the trunk portion of the expandable branching stent prosthesis and the first branching portion of the expandable branching stent prosthesis for deployment. In some embodiments, inflating 710 the first balloon includes inflating a first balloon portion of the first balloon to a first diameter or a first lateral width to expand the first branching portion of the expandable branch stent prosthesis and also inflating a trunk balloon portion of the first balloon to a trunk diameter or the trunk lateral width that is greater than the first diameter or the first lateral width of the first balloon portion to expand the trunk portion of the expandable branching stent prosthesis.

The method 700 further includes inflating 712 the second balloon to expand the second branching portion of the expandable branching stent prosthesis for deployment. In some embodiments, inflating 712 the second balloon includes inflating 712 the second balloon with a second catheter coupled to the second balloon and having a second inflation lumen to expand the second branching portion of the expandable branching stent prosthesis for deployment. In the method 700, the second catheter may be disposed outside the first balloon. In some embodiments of the method 700, the second catheter includes the second inflation lumen, an additional inflation lumen in fluid communication with the second balloon and configured to selectively inflate the second balloon, and a wire lumen having the floss wire extending therethrough until the floss wire is pulled through the second branch and out of the access incision of the second branch. In some embodiments of the method 700, the floss wire extends through the second balloon until the floss wire is pulled through the second branch and out of the access incision of the second branch. In some embodiments, inflating 712 the second balloon includes inflating the second balloon to a second diameter or second lateral width that is less than the trunk diameter or the trunk lateral width of the trunk balloon portion

In some embodiments, inflating 710 the first balloon and inflating 712 the second balloon may be substantially simultaneous. For example, the second balloon may be separate and distinct from the first balloon, but the inflation lumens of the first catheter and the second catheter may be fluidly connected or coupled such that inflating 710 the first balloon and inflating 712 the second balloon may be substantially simultaneous. In some embodiments, inflating 710 the first balloon and inflating 712 the second balloon may not be simultaneous (e.g. may be at different times). For example, the inflation lumens of the first catheter and the second catheter are separate may be separate, and the first balloon and the second balloon may be inflated at different times.

In some embodiments, the method 700 further includes, after inflating the first balloon and the second balloon, pulling the snare wire to bring the distal end of the floss wire through the second branch and out of an access incision of the second branch. In some embodiments, the method 700 further includes deflating the first balloon, deflating the second balloon, and, after pulling the snare wire to bring the distal end of the floss wire through the second branch, removing the deployment system from the branching implant site by pulling the first catheter and/or the second catheter such that the second balloon is pulled from the second branch, over a bifurcation between the second branch and the first branch, and through the first branch. In some embodiments, the first catheter and second catheter are connected such pulling one catheter (of the first catheter and the second catheter) the other catheter (of the first catheter and the second catheter) is simultaneously pulled for removal from the branching implant site. In some embodiments, the second catheter may not be connected to the first catheter at any point along first catheter, or may be selectively connected and disconnectable. When the second catheter is disconnected from the first catheter, the first catheter and the second catheter may be pulled separately and/or non-simultaneously (e.g., at different times) from the implant site, thus requiring a lower removal force.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A deployment system for deployment of a device to a branching implant site having a trunk, a first branch, and a second branch, the deployment system comprising:
an expandable branching stent prosthesis having a trunk portion, a first branching portion, and a second branching portion;
a first balloon disposed within the first branching portion and the trunk portion, the first balloon being configured to inflate to deploy the first branching portion and the trunk portion of the expandable branching stent prosthesis;
a first catheter coupled to the first balloon and having a first inflation lumen, and the first catheter configured to selectively inflate the first balloon;
a second balloon disposed within the second branching portion and configured to inflate to deploy the second branching portion of the expandable branching stent prosthesis; and
a second catheter coupled to the second balloon and having a second inflation lumen, the second catheter configured to selectively inflate the second balloon.

2. The deployment system of claim 1, further comprising a floss wire passing through the first branching portion and out the second branching portion of the expandable branching stent prosthesis, the floss wire having a distal end disposed outside of the second branching portion of the expandable branching stent prosthesis.

3. The deployment system of claim 1, wherein the second catheter is disposed outside the first balloon.

4. The deployment system of claim 1, wherein the second catheter includes:
the second inflation lumen and an additional inflation lumen in fluid communication with the second balloon and configured to selectively inflate the second balloon; and
a wire lumen having the floss wire extending therethrough.

5. The deployment system of claim 1, wherein the floss wire extends through the second balloon.

6. The deployment system of claim 1, wherein the distal end of the floss wire terminates in a catch.

7. The deployment system of claim 1, wherein, when inflated, the first balloon includes:
a first balloon portion having a first diameter or a first lateral width and disposed in the first branching portion; and
a trunk balloon portion having a trunk diameter or a trunk lateral width greater than the first diameter or the first lateral width of first balloon portion, with the trunk balloon portion being disposed in the trunk portion of the expandable branching stent prosthesis.

8. The deployment system of claim 1, wherein the first inflation lumen and the second inflation lumen are fluidly coupled such that the first balloon and the second balloon are configured to inflate and/or deflate substantially simultaneously.

9. The deployment system of claim 1, wherein the first inflation lumen and the second inflation lumen are not fluidly coupled such that the first balloon and the second balloon are configured to selectively inflate and/or deflate substantially simultaneously or at different times.

10. A method comprising:
delivering, to a branching implant site comprising a trunk, a first branch, and a second branch, a deployment system via the first branch, the deployment system comprising:
an expandable branching stent prosthesis having a trunk portion, a first branching portion, and a second branching portion;
a first balloon disposed within the first branching portion and the trunk portion of the expandable branching stent prosthesis;
a second balloon disposed within the second branching portion; and
a floss wire passing through the first branching portion and out the second branching portion of the expandable branching stent prosthesis, the floss wire having a distal end disposed outside of the second branching portion of the expandable branching stent prosthesis;
delivering a distal end of a snare wire to the branching implant site via the second branch, the snare wire extending back through the second branch;
connecting the distal end of the floss wire and the distal end of the snare wire together at the branching implant site;
using one or more of the floss wire and the snare wire to seat the first branching portion of the expandable branching stent prosthesis along the first branch of the branching implant site and the second branching portion of the expandable branching stent prosthesis along the second branch of the branching implant site;
inflating the first balloon to expand the trunk portion of the expandable branching stent prosthesis and the first branching portion of the expandable branching stent prosthesis for deployment; and
inflating the second balloon to expand the second branching portion of the expandable branching stent prosthesis for deployment.

11. The method of claim 10, further comprising, after inflating the first balloon and the second balloon, pulling the snare wire to bring the distal end of the floss wire through the second branch and out of an access incision of the second branch.

12. The method of claim 10, wherein:
inflating the first balloon to expand the trunk portion of the expandable branching stent prosthesis and the first branching portion of the expandable branching stent prosthesis for deployment includes:
inflating the first balloon with a first catheter coupled to the first balloon and having a first inflation lumen in fluid communication with the first balloon to expand the trunk portion of the expandable branching stent prosthesis and the first branching portion of the expandable branching stent prosthesis for deployment; and
inflating the second balloon to expand the second branching portion of the expandable branching stent prosthesis for deployment includes:
inflating the second balloon with a second catheter coupled to the second balloon and having a second inflation lumen to expand the second branching portion of the expandable branching stent prosthesis for deployment.

13. The method of claim 10, wherein the second catheter includes:
the second inflation lumen and an additional inflation lumen in fluid communication with the second balloon and configured to selectively inflate the second balloon; and
a wire lumen having the floss wire extending therethrough until the floss wire is pulled through the second branch and out of the access incision of the second branch.

14. The method of claim 13, wherein the floss wire extends through the second balloon until the floss wire is pulled through the second branch and out of the access incision of the second branch.

15. The method claim 13, further comprising:
deflating the first balloon and the second balloon; and
after pulling the snare wire to bring the distal end of the floss wire through the second branch, removing the deployment system from the branching implant site by pulling the first catheter and/or the second catheter such that the second balloon is pulled from the second branch, over a bifurcation between the second branch and the first branch, and through the first branch.

16. The method of claim 15, further comprising selectively disconnecting the first catheter from the second catheter before removing the deployment system from the branching implant site, wherein removing the deployment system from the branching implant site by pulling the first catheter and/or the second catheter includes pulling the first catheter to remove the first balloon and pulling the second catheter separate from the first catheter to remove the second balloon.

17. The method of claim 15, wherein the first catheter is selectively or fixedly connected to the second catheter such that removing the deployment system from the branching implant site by pulling the first catheter and/or the second catheter includes removing the deployment system from the branching implant site by pulling either one of the first catheter or the second catheter to pull the first balloon from the first branch and pull the second balloon from the second branch, over the bifurcation between the second branch and the first branch, and through the first branch.

18. The method of claim 10, wherein connecting the distal end of the floss wire and the distal end of the snare wire together at the branching implant site includes connecting the distal end of the snare wire to a catch at the distal end of the floss wire.

19. The method of claim 10, wherein inflating the first balloon to expand the trunk portion of the expandable branching stent prosthesis and the first branching portion of the expandable branching stent prosthesis for deployment includes:
inflating a first balloon portion of the first balloon to a first diameter or a first lateral width to expand the first branching portion of the expandable branch stent prosthesis; and
inflating a trunk balloon portion of the first balloon to a trunk diameter or the trunk lateral width that is greater than the first diameter or the first lateral width of the first balloon portion to expand the trunk portion of the expandable branching stent prosthesis.

20. The method of claim 19, wherein inflating the second balloon to expand the second branching portion of the expandable branching stent prosthesis for deployment includes:
inflating the second balloon to a second diameter or second lateral width that is less than the trunk diameter or the trunk lateral width of the trunk balloon portion.
